(19) United States
(12) Patent Application Publication
(10) Pub. No.: US 2004/0037784 A1
(43) Pub. Date: Feb. 26, 2004

(54) Dry-powder pharmaceutical formulation for inhalation comprising alpha4-integrin antagonist

(76) Inventors: Duncan Robert Armour, Stevenage Hertfordshire (GB); David Brown, Stevenage Hertfordshire (GB); Miles Stuart Congreve, Stevenage Hertfordshire (GB); Paul Martin Gore, Stevenage Hertfordshire (GB); Darren Victor Steven Green, Stevenage Hertfordshire (GB); Stuart Holman, Stevenage Hertfordshire (GB); Torquil Iain Maclean Jack, Stevenage Hertfordshire (GB); Steven Philip Keeling, Stevenage Hertfordshire (GB); Andrew McMurtrie Mason, Stevenage Hertfordshire (GB); Karen Morris, Stevenage Hertfordshire (GB); Nigel Grahame Ramsden, Stevenage Hertfordshire (GB); Marian Thomas, Ware Hertfordshire (GB); Peter Ward, Stevenage Hertfordshire (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/311,558

(22) PCT Filed: Jun. 15, 2001
(86) PCT No.: PCT/GB01/02656

(30) Foreign Application Priority Data
Jun. 16, 2000 (GB) 0014851.0

(51) Int. Cl. A61L 9/04; A61K 9/14; A61K 31/445

(52) U.S. Cl. 424/46; 514/53; 514/317

(57) ABSTRACT

The present invention relates to a dry powder pharmaceutical formulation suitable for inhalation therapy comprising particulate (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof and one or more suitable excipients. Methods and uses of the formulation in the treatment of inflammatory diseases are also described, as are medicament packs and inhalation devices containing said formulation.

The present invention relates to dry powder formulations for use in the treatment of respiratory disorders by inhalation.

Numerous medicaments, especially those for the treatment of respiratory conditions such as asthma or chronic obstructive pulmonary disease (COPD), are administered by dry powder inhalation. Since the drug acts directly on the target organ much smaller quantities of the active ingredient may be used, thereby minimising any potential side effects caused as a result of systemic absorption. The delivery systems currently available for powder formulations include dry powder inhalers.

(2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid has recently been described in International Patent Application PCT/EP99/10000 as a novel antagonist of both α₄β₁, and α₄β₇ integrins which, as a consequence, results in effective anti-inflammatory properties. However, there is a need for a dry powder formulation suitable for treatment of inflammatory conditions, in particular asthma.

Excipients are particularly necessary for dry powder inhalation formulations, especially if the efficacy of the pharmaceutical substance is very high, to ensure that only small amounts of medicament are delivered in each dose. In such a scenario it is necessary to dilute the active substance so as to achieve good accuracy of metering

Thus, according to the present invention we provide a dry powder pharmaceutical formulation suitable for inhalation therapy comprising particulate (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof and one or more suitable excipients.

Examples of suitable salts include physiologically acceptable salts such as alkali metal salts, for example calcium, sodium and potassium salts and salts with (trishydroxymethyl)aminomethane.

Preferably, the (2S)-3-[4-({[4-(Aminocarbonyl)-1-piperidinyl]carbonyl}oxy) phenyl]-2-[((2S)4-methyl-2-{[2-(2-methylphenoxy)acetyl]amino}pentanoyl) amino] propanoic acid will be present as the potassium salt.

Also preferably, the (2S)-3-[4-({[4-(Aminocarbonyl)-1-piperidinyl]carbonyl}oxy) phenyl]-2-[((2S)-4-methyl-2-{[2-(2-methylphenoxy)acetyl]amino}pentanoyl) amino] propanoic acid will be present as the free acid.

Such excipients must be physiologically acceptable when used in administration by the aerial pathways.

The excipients which satisfy this requirement will be selected from a group comprising monosaccharides (eg. glucose and arabinose), disaccharides (eg. lactose, saccharose and maltose), polysaccharides (eg. sorbitol, mannitol and xylitol), salts (eg. sodium chloride and calcium carbonate), amino acids, peptides, polymers, lipids or a mixture thereof.

Preferably, the excipients are mono-, di- or polysaccharides, among which, use is preferably made of one of the two diglucosides lactose and trehalose, especially lactose.

Preferably, the formulation will contain between 0.1 mg and 10 mg of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof, especially between 0.5 mg and 5 mg per dose.

Preferably, the formulation will contain between 0.1 mg and 25 mg excipient, most preferably between 20 mg and 24.5 mg per dose or between 7.5 mg and 12 mg per dose.

Such a high proportion of excipient will essentially determine the properties of the powder formulation, particularly the flow characteristics. Generally, if the powder is fine then the flow properties will be poor. Since good flow properties are the prerequisite for good accuracy of metering when filling individual containers with a prepared dose eg. when preparing capsules for powder inhalation in conventional capsule machines, the excipient used must not be too fine. Similarly the particle size of the powder must not be too coarse as this will effect the expelling and dispersing characteristics from a dry powder inhaler.

Optionally a further particulate active ingredient suitable for inhalation therapy may be incorporated into the formulation such as a corticosteroid (eg fluticasone propionate) or a bronchodilator (eg salmeterol or albuterol or a salt thereof).

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways.

Therefore, we prefer that the mass median diameter (MMD) of the (2S)-3-[4-({[4-(Aminocarbonyl)-1-piperidinyl]carbonyl}oxy)phenyl]-2-[((2S)4-methyl-2-{[2-(2-methylphenoxy)acetyllamino}pentanoyl)amino] propanoic acid or a salt or solvate thereof is between 1 and 10 μm, most preferably between 2 and 5 μm.

To achieve these particle sizes the particles of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof as produced may be size reduced by conventional means eg. by micronisation. The desired fraction may be separated out by air classification or sieving. Preferably, the particles will be crystalline, prepared for example by a process which comprises mixing in a continuous flow cell in the presence of ultrasonic radiation a flowing solution of (2S)-3-[4-({[4 (Aminocarbonyl)-1-piperidinyl]carbonyl}oxy) phenyl]-2-[((2S)4-methyl-2-{[2-(2-methylphenoxy)acetyl]amino}pentanoyl) amino] propanoic acid (or a salt or solvate thereof) as medicament in a liquid solvent with a flowing liquid antisolvent for said medicament (as described in International Patent Application PCT/GB99/04368).

Generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

It is now possible to control the proportion of inhaled medicaments while retaining a good accuracy for metering. This may be achieved by combining a medicament which has been micronised into inhalable particles (eg. less than 10μ) with suitable quantities of a mixture of one or more physiologically acceptable excipients, one component of which has a particle size of less than 15 μm-and the other which has a mean particle size greater than about 20 μm (generally below 150 μm, preferably below 80 μm).

Therefore, we prefer that one component of the excipient has a MMD of less than 15 μm (the fine excipient component) and another component of the excipient has a MMD of greater than 20 μm but lower than 150 μm, preferably lower than 80 μm (the coarser excipient component).

The excipient or excipients may be commercially available in the desired particle size range or may also be separated by air classification or sieving.

Preferably, the weight ratio of the fine and coarser excipient components will range from 10:90 to 50:50.

Finely divided and coarser excipients may consist of chemically identical or chemically different substances. The excipient mixtures may, for example, contain one chemical substance as the fine excipient and a different substance as the coarser excipient. However, the fine and coarser excipients in question may also themselves constitute mixtures of different substances. Preferably, the fine and coarser excipients will both be lactose.

It will be appreciated that the formulations according to the present invention may contain minor amounts of other additives eg. taste masking agents or sweeteners.

Packaging of the formulation may be suitable for unit dose or multi-dose delivery. In the case of multi-dose delivery, the formulation can be pre-metered (eg. as in Diskus, see GB 2242134 or Diskhaler, see GB 2178965, 2129691 and 2169265) or metered in use (eg. as in Turbuhaler, see EP 69715). An example of a unit-dose device is Rotahaler (see GB 2064336).

As a particular aspect of the present invention we also provide a medicament pack for use in an inhalation device which comprises an elongate strip formed from a base sheet having a plurality of recesses spaced along its length and a lid sheet hermetically but peelably sealed thereto to define a plurality of containers, each container having therein an inhalable formulation according to the present invention.

Preferably, the strip is sufficiently flexible to be wound into a roll.

The lid sheet and base sheet will preferably have leading end portions which are not sealed to one another and at least one of the said leading end portions is constructed to be attached to a winding means.

Also, preferably the hermetic seal between the base and lid sheets extends over their whole width. The lid sheet may preferably be peeled from the base sheet in a longitudinal direction from a first end of the said base sheet.

Such a peelable blister strip is preferably suitable for administration using a Diskus inhaler eg as described in GB 2242134.

As a further aspect of the present invention we also provide an inhalation device for use with a medicament pack which comprises a formulation according to the present invention, said device comprising:

(i) an opening station for receiving a container of a medicament pack being used with said inhalation device;

(ii) means positioned to engage peelable sheets of a container which has been received in said opening station for peeling apart the peelable sheets, to open such a container;

(iii) an outlet, positioned to be in communication with an opened container, through which a user can inhale medicament in powder form from such an opened container; and

(iv) indexing means for indexing in communication with said outlet containers of a medicament pack in use with said inhalation device.

As an alternative aspect of the present invention we also provide a medicament pack comprising a circular carrier disc which has a plurality of pre-filled, hermetically sealed containers formed integrally therewith and arranged in a circle, each container containing an inhalable formulation according to the present invention, each container being puncturable to form a hole on each side thereof to allow in use, air to flow through the container to entrain the powder contained therein.

As a further aspect of the present invention there is also provided an inhalation device by which formulations of the present invention may be administered to a patient which comprises a housing, a tray mounted and capable of moving within said housing (via a plunger) adapted to receive a circular carrier disc medicament pack, an air inlet (through which air can enter said device) and an air outlet (through which a patient may inhale and receive said formulation.

As an alternative aspect of the present invention we also provide a medicament pack comprising a piercable capsule which contains a formulation according to the present invention.

As a further aspect of the present invention there is also provided an inhalation device by which formulations of the present invention may be administered to a patient which comprises a body shell which has a nozzle at a forward end and which is open at the rear end, a sleeve fitted on the outside of the body shell and rotatable with respect to it, a means for retaining a piercable capsule extending through the rear wall of the sleeve into the body shell, means for piercing said capsule when sleeve is rotated and a guard to ensure that the formulation and not the pierced capsule, passes through the nozzle.

As a further aspect of the present invention there is also provided an inhalation device by which formulations of the present invention may be administered to a patient which comprises a nozzle, an air conduit connected to said nozzle for allowing a passage of air to be inhaled, a dosing unit comprising a storage chamber for the formulation (which may also comprise a dosage indicating means) and a displaceable element for dispensing said formulation from the storage chamber into the air conduit, a manoeuvering unit for displacing said element in relation to the storage chamber and optional deflector devices to provide accelerated airflow.

In a further or alternative aspect of the present invention, there is provided a method of treatment or prophylaxis of inflammatory diseases which comprises administering to a patient by inhalation a formulation according to the present invention.

A suitable dosing regime for the formulation of the present invention would be the delivery of between 0.1 and 10 mg of the (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid (or a salt or solvate thereof), preferably between 0.5 and 5 mg.

Typically, 1 or 2 inhalations would be administered by the above procedure up to three times each day.

Preferably, the inflammatory disease will be asthma.

According to another aspect of the present invention there is provided the use of a formulation according to the present invention in the manufacture of a medicament for the treatment of inflammatory diseases by inhalation.

Above mentioned patents and patent applications are hereinbefore incorporated by reference.

The invention may be illustrated by the following non-limiting examples:

### EXAMPLE A

### (2S)-3-[4-({[4(Aminocarbonyl)-1-piperidinyl]carbonyl} oxy) phenyl]-2-[((2S)4-methyl-2-{[2-(2-methylphenoxy)acetyl]amino}pentanoyl) amino] Propanoic Acid

To Wang resin (50 g) was added a solution of (2S)-3-[4-(allyloxy)phenyl]-2-[(tert-butoxycarbonyl)amino]propanoic acid (115.8 g) and 1-hydroxybenzotriazole (48.6 g) in DMF (475 ml). After 15 minutes 1,3-diisopropylcarbodiimide (56.5 ml) was added and the mixture was stirred for 24 h at 45° C. The resin was filtered and washed with DMF (3×360 ml), methanol (3×360 ml) and dichloromethane (3×700 ml). To a slurry of the resin in dichloromethane (644 ml) was added pyridine (14.7 ml). Acetic anhydride (26.9 ml) was added and the mixture was stirred for 12 h at 20° C. The resin was filtered and washed with dichloromethane (3×550 ml), methanol (3×370 ml) and dichloromethane (3×550 ml).

A slurry of 20 g of the resin in dichloromethane (100 ml) was cooled to 2-5° C. and treated with a solution of phenol (20 g) in dichloromethane (80 ml). Chlorotrimethylsilane (20 ml) was added dropwise and the mixture was stirred for 6 h at 2-5° C. The resin was filtered and washed with dichloromethane (3×200 ml), methanol (3×200 ml), 10% water in DMF (2×200 ml), 10% diisopropylethylamine in DMF (3×200 ml), DMF (200 ml), methanol (3×200 ml) and dichloromethane (3×200 ml).

A slurry of the resin in DMF (55 ml) was treated with a solution of Fmoc-leucine (32.7 g) and 1-hydroxybenzotriazole (12.5 g) in DMF (85 ml). After 5 minutes 1,3-diisopropylcarbodiimide (19.3 ml) was added and the mixture was stirred for 15 h at 20° C. The resin was filtered and washed with DMF (3×150 ml), methanol (3×150 ml) and dichloromethane (3×150 ml).

The resin was treated with 20% piperidine in DMF (180 ml) and stirred for 1h at 20° C. The resin was filtered and washed with DMF (3×150 ml), dichloromethane (3×150 ml), DMF (3×150 ml) and dichloromethane (3×150 ml). To a slurry of this in DMF (50 ml) was added a solution of (2-methylphenoxy)acetic acid (17.9 g) and 1-hydroxybenzotriazole (14.6 g) in DMF (100 ml). After 0.5 minutes 1,3-diisopropylcarbodiimide (16.9 ml) was added and the mixture was stirred for 65 h at 20° C. The resin was filtered and washed with DMF (2×150 ml), methanol (3×150 ml) and dichloromethane (3×150 ml).

A slurry of the resin in dichloromethane (60 ml) was treated with a solution of tetrakis(triphenylphosphine)palladium(0) (5.21 g) in dichloromethane (140 ml) followed by morpholine (13 ml). The mixture was stirred for 2 h at 20° C. then the resin was filtered and washed with dichloromethane (7×200 ml).

A slurry of the resin in dichloromethane (160 ml) was treated with diisopropylethylamine (12.4 ml) followed by 4-nitrophenyl chloroformate (24.8 g) in 3 portions at 5 minute intervals. The mixture was stirred for 1 h at 20° C. The resin was filtered and washed with dichloromethane (3×200 ml).

The resin was treated with a solution of isonipecotamide (15.8 g) in DMF (180 ml) and the mixture was stirred for 1.5 h at 20° C. The resin was filtered and washed with DMF (4×200 ml) and dichloromethane (2×200 ml).

The resin was treated with 50% TFA in dichloromethane (200 ml). After stirring for 1 h at 20° C. the resin was filtered and washed with dichloromethane (5×200 ml). The combined filtrate and washings were evaporated in vacuo. The residue was azeotroped with toluene (2×100 ml) then triturated with ether (50 ml) and the resulting white solid filtered. To this was added acetonitrile (150 ml) and the mixture was heated to reflux. The resulting suspension was allowed to cool to 20° C. and stirred for 18 h. The mixture was filtered to give the title compound as a white solid (4.9 g).

### EXAMPLE B

### (2S)-3-[4-({[4-(Aminocarbonyl)-1-piperidinyl]carbonyl} oxy) phenyl]-2-[((2S)4-methyl-2{2-(2-methylphenoxy)acetyl]amino}pentanoyl) amino] Propanoic Acid Potassium Salt

A suspension of Example A (10 g) in methanol (150 ml) was warmed to reflux to obtain a clear solution. To this was added a solution of potassium carbonate (1.16 g) in water (7.5 ml). After heating under reflux for two minutes the solvents were evaporated in vacuo to give a crisp foam. To this was added acetonitrile (100 ml) and the mixture was warmed to reflux, during which time the foam collapsed and started to crystallise. After ten minutes the mixture was allowed to cool to 20° C. then filtered under reduced pressure, washed with acetonitrile (25 ml) and ether (50 ml) to give the title compound as a white solid (10.65 g, 100%).

### EXAMPLE C

| | |
|---|---|
| (2S)-3-[4-({[-(Aminocarbonyl)-1-piperidinyl]carbonyl}oxy)- | 0.5 mg |
| phenyl]-2-[((2S)-4-methyl-2-{[2-(2-methylphenoxy)acetyl]- | |
| amino}pentanoyl)amino] propanoic acid (prepared according to | |
| Example A and micronised to a MMD of 3 μm): | |
| milled lactose (wherein not greater than 85% of particles have a | 24.5 mg |
| mean diameter of 60-90 μm, and not less than 15% of part- | |
| icles have a mean diameter of less than 15 μm): | |

### EXAMPLE D

| | |
|---|---|
| (2S)-3-[4-({[4-(Aminocarbonyl)-1-piperidinyl]carbonyl}oxy)- | 5 mg |
| phenyl]-2-[((2S)-4-methyl-2-{[2-(2-methylphenoxy)acetyl]- | |
| amino}pentamoyl)amino] propanoic acid potassium salt (prepar- | |
| ed according to Example B and micronised to a MMD of 3 μm): | |
| milled lactose (wherein not greater than 85% of particles have a | 20 mg |
| mean diameter of 60-90 μm, and not less than 15% of particles | |
| have a mean diameter of less than 15 μm): | |

### EXAMPLE E

| | |
|---|---|
| (2S)-3-[4-({[4-(Aminocarbonyl)-1-piperidinyl]carbonyl}oxy)- | 0.5 mg |
| phenyl]-2-[((2S)-4-methyl-2-{[2-(2-methylphenoxy)acetyl]- | |
| amino}pentanoyl)amino]propanoic acid (prepared according to | |
| Example A and micronised to a MMD of 3 μm): | |
| milled lactose (wherein not greater than 85% of particles | 12 mg |
| have a MMD of 60-90 μm, and not less than 15% of particles | |
| have a MMD of less than 15 μm): | |

### EXAMPLE F

| | |
|---|---|
| (2S)-3-[4-({[4-(Aminocarbonyl)-1-piperidinyl]carbonyl}oxy)- | 1 mg |
| phenyl]-2-[((2S)-4-methyl-2-{[2-(2-methylphenoxy)acetyl]- | |
| amino}pentanoyl)amino] propanoic acid potassium salt | |
| (prepared according to Example B and micronised to a | |
| MMD of 3 μm): | |
| milled lactose (wherein not greater than 85% of particles have a | 11.5 mg |
| mean diameter of 60-90 μm, and not less than 15% of particles | |
| have a mean diameter of less than 15 μm): | |

### EXAMPLES 1-2

A peelable blister strip containing 60 blisters each filled with a formulation according to Examples C-D.

### EXAMPLES 3-4

A peelable blister strip containing 60 blisters each filled with a formulation according to Examples E-F.

### EXAMPLES 5-6

A circular carrier disk having 4 hermetically sealed and puncturable containers each filled with a formulation according to Examples C-D arranged in a circular carrier disc (eg. Rotadisk™).

### EXAMPLES 7-8

A piercable capsule filled with a formulation according to Examples C-D (eg. Rotacap™.)

### EXAMPLE 9

A bulk formulation which contains:

| | |
|---|---|
| (2S)-3-[4-({[4-(Aminocarbonyl)-1-piperidinyl]carbonyl}oxy)- | 20 mg |
| phenyl]-2-[((2S)-4-methyl-2-{[2-(2-methylphenoxy)acetyl]- | |
| amino}pentanoyl)amino] propanoic acid potassium salt | |
| (prepared according to Example A and micronised to a MMD | |
| of 3 μm): | |
| milled lactose (wherein not greater than 85% of particles | 980 mg |
| have a MMD of 60-90 μm, and not less than 15% of particles | |
| have a MMD of less than 15 μm): | |

suitable for filling into a multi-dose inhaler which meters in use (eg. Turbuhaler™).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A dry powder pharmaceutical formulation suitable for inhalation therapy comprising particulate (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof and one or more suitable excipients.

2. A pharmaceutical formulation according to claim 1 wherein the (2S)-3-[4-({[4-(Aminocarbonyl)-1-piperidinyl]carbonyl}oxy)phenyl]-2-[((2S)-4-methyl-2-[{2-(2-methylphenoxy)acetyl]amino}pentanoyl)amino] propanoic acid is present as the potassium salt.

3. A pharmaceutical formulation according to claim 1 wherein the (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid is present as the free acid.

4. A pharmaceutical formulation according to claim 1 wherein the excipients are selected from a group comprising monosaccharides (eg. glucose and arabinose), disaccharides (eg. lactose, saccharose and maltose), polysaccharides (eg. sorbitol, mannitol and xylitol), salts (eg. sodium chloride and calcium carbonate), amino acids, peptides, polymers, lipids or a mixture thereof.

5. A pharmaceutical formulation according to claim 4 wherein the excipient is a mono-, di- or polysaccharide

6. A pharmaceutical formulation according to claim 5 wherein the excipient is lactose or trehalose.

7. A pharmaceutical formulation according to claim 6 wherein the excipient is lactose.

8. A pharmaceutical formulation according to any one of claims 1 to 7 wherein said formulation contains between 0.1 mg and 10 mg of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof per dose.

9. A pharmaceutical formulation according to claim 8 wherein said formulation contains between 0.5 mg and 5 mg of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof per dose.

10. A pharmaceutical formulation according to any one of claims 1 to 9 wherein said formulation contains between 0.1 mg and 25 mg excipient.

11. A pharmaceutical formulation according to claim 10 wherein said formulation contains between 20 mg and 24.5 mg excipient or between 7.5 mg and 12 mg excipient.

12. A pharmaceutical formulation according to any one of claims 1 to 11 wherein the MMD of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl)amino] propanoic acid or a salt or solvate thereof is between 1 and 10 μm.

13. A pharmaceutical formulation according to claim 12 wherein the MMD of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof is between 2 and 5 μm.

14. A pharmaceutical formulation according to any one of claims 1 to 13 wherein one component of the excipient has a MMD of less than 15 μm (the fine excipient component) and another component of the excipient has a MMD of greater than 20 μm but lower than 150 μm (the coarser excipient component).

15. A pharmaceutical formulation according to claim 14 wherein one component of the excipient has a MMD of less than 15 μm (the fine excipient component) and another component of the excipient has a MMD of greater than 20 μm but lower than 80 μm (the coarser excipient component).

16. A pharmaceutical formulation according to claim 14 or claim 15 wherein the weight ratio of the fine and coarser excipient components will range from 10:90 to 50:50.

17. A pharmaceutical formulation according to any one of claims 14 to 16 wherein the fine and coarser excipients are both lactose.

18. A medicament pack for use in an inhalation device which comprises an elongate strip formed from a base sheet having a plurality of recesses spaced along its length and a lid sheet hermetically but peelably sealed thereto to define a plurality of containers, each container having therein an inhalable formulation according to any one of claims 1 to 17.

19. A medicament pack according to claim 18 wherein the strip is sufficiently flexible to be wound into a roll.

20. A medicament pack according to claim 18 wherein the lid sheet and base sheet have leading end portions which are not sealed to one another.

21. A medicament pack according to claim 20 wherein at least one of the said leading end portions is constructed to be attached to a winding means.

22. A medicament pack according to claim 18 wherein the hermetic seal between the base and lid sheets extends over their whole width.

23. A medicament pack according to claim 18 wherein the lid sheet may be peeled from the base sheet in a longitudinal direction from a first end of the said base sheet.

24. An inhalation device for use with a medicament pack according to any one of claims 18 to 23 which comprises a formulation according to any one of claims 1 to 17, said device comprising:
(i) an opening station for receiving a container of a medicament pack being used with said inhalation device;
(ii) means positioned to engage peelable sheets of a container which has been received in said opening station for peeling apart the peelable sheets, to open such a container;
(iii) an outlet, positioned to be in communication with an opened container, through which a user can inhale medicament in powder form from such an opened container; and
(iv) indexing means for indexing in communication with said outlet containers of a medicament pack in use with said inhalation device.

25. A medicament pack comprising a circular carrier disc which has a plurality of pre-filled, hermetically sealed containers formed integrally therewith and arranged in a circle, each container containing an inhalable formulation according to any one of claims 1 to 17, each container being puncturable to form a hole on each side thereof to allow in use, air to flow through the container to entrain the powder contained therein.

26. An inhalation device by which formulations according to any one of claims 1 to 17 may be administered to a patient which comprises a housing, a tray mounted and capable of moving within said housing (via a plunger) adapted to receive a circular carrier disc medicament pack according to claim 25, an air inlet (through which air can enter said device) and an air outlet (through which a patient may inhale and receive said formulation.

27. A medicament pack comprising a piercable capsule which contains a formulation according to any one of claims 1 to 17.

28. An inhalation device by which formulations according to any one of claims 1 to 17 may be administered to a patient which comprises a body shell which has a nozzle at a forward end and which is open at the rear end, a sleeve fitted on the outside of the body shell and rotatable with respect to it, a means for retaining a piercable capsule according to claim 27 extending through the rear wall of the sleeve into the body shell, means for piercing said capsule when sleeve is rotated and a guard to ensure that the formulation and not the pierced capsule, passes through the nozzle.

29. An inhalation device by which formulations according to any one of claims 1 to 17 may be administered to a patient which comprises a nozzle, an air conduit connected to said nozzle for allowing a passage of air to be inhaled, a dosing unit comprising a storage chamber for the formulation (which may also comprise a dosage indicating means) and a displaceable element for dispensing said formulation from the storage chamber into the air conduit, a manoeuvering unit for displacing said element in relation to the storage chamber and optional deflector devices to provide accelerated airflow.

30. A method of treatment or prophylaxis of inflammatory diseases which comprises administering by inhalation to a patient a formulation according to any one of claims 1 to 17.

31. A method of treatment or prophylaxis of asthma which comprises administering by inhalation to a patient a formulation according to any one of claims 1 to 17.

32. Use of a formulation according to any one of claims 1 to 17 in the manufacture of a medicament for the treatment of inflammatory diseases by inhalation.

33. Use of a formulation according to any one of claims 1 to 17 in the manufacture of a medicament for the treatment of asthma by inhalation.
